(19) United States
(12) Patent Application Publication
(10) Pub. No.: US 2010/0324413 A1
(43) Pub. Date: Dec. 23, 2010

(54) X-RAY DIAGNOSTIC SYSTEM

(75) Inventors: Akio Tetsuka, Tochigi-ken (JP); Shingo Abe, Tochigi-ken (JP); Kunio Shiraishi, Tochigi-ken (JP)

Correspondence Address: OBLON, SPIVAK, MCCLELLAND MAIER & NEUSTADT, L.L.P., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/797,040

(22) Filed: Jun. 9, 2010

(30) Foreign Application Priority Data
Jun. 22, 2009 (JP) 2009-147563

(51) Int. Cl. A61B 6/12

(52) U.S. Cl. 600/424

(57) ABSTRACT

In one embodiment, an X-ray diagnostic system is provided. The system is provided with an X-ray irradiation section, an X-ray detection section, an image data generation unit, a position detecting unit and a control section. The X-ray irradiation section radiates an X-ray to an object to be diagnosed. The X-ray detection section detects the X-ray radiated from the X-ray irradiation section and transmitted through the object so as to generate X-ray transmission data. The image data generation unit generates image data based on the X-ray transmission data. The position detecting unit detects position of a catheter inserted into the object. The control section switches X-ray irradiation conditions to be irradiated from the X-ray irradiation section, based on positional data detected by the position detecting unit and electrocardiogram data of the object.

### CROSS REFERENCE TO RELATED APPLICATION

### Quotation of Related Application

This application is based upon and claims the benefit of priority from the prior Japanese Patent Application No. 2009-147563, filed on Jun. 22, 2009, the entire contents of which are incorporated herein by reference.

### FIELD

Embodiments described herein relate generally to an X-ray diagnostic system.

### BACKGROUND

Recent years, an X-ray diagnostic imaging system has progressed mainly in a circulatory organ field, with the development of an angiographic examination using a catheter or an IVR (Interventional Radiology). In the circulatory organ field, the X-ray diagnostic imaging system is used to perform a diagnosis or medical treatment of a blood vessel system existing in a site such as a heart, a head, an abdomen or a limb.

Contraction and expansion of a heart being repeated regularly are caused by electric stimuli. Pulsation rhythm of a heart become irregular and an arrhythmic symptom occur, when abnormality exists in a conduction path of the heart through which an electric stimulus is transmitted.

JP2005-253801-A discloses a method of performing a therapy for an arrhythmia of a heart. The disclosed therapeutic method utilizes an ablation technique. According to the method, an X-ray is irradiated to an object to be examined from an X-ray irradiation section of an X-ray-diagnostic system. An operator performs burning off an abnormal conduction path of a heart while watching image data generated as a result of the irradiation.

In the therapy, after the operator inserts a catheter for ablation from a blood vessel of the object, he moves a tip portion of the catheter to the heart so that it reaches the heart. The operator burns off the abnormal conduction path by providing a high frequency current to an electrode arranged in the tip portion, under the state where the abnormal conduction path of the heart is in contact with the tip portion.

Such a medical treatment needs to irradiate X-ray to the object for a long time, because it is necessary to check whether the tip portion of the catheter is at a suitable position of an inner wall of the heart after insertion of the catheter into the object. As a result, the object can be irradiated badly.

### BRIEF DESCRIPTION OF THE DRAWINGS

FIG. 1 is a block diagram showing a configuration of a system to diagnose and to perform therapy using an X-ray according to an embodiment.

FIG. 2 shows an example of an arm movement mechanism provided in the system of the embodiment.

FIG. 3 shows an arrangement of a catheter to be used in the system of the embodiment.

FIG. 4 shows a view where a tip portion of the catheter is in contact with an inner wall of an atrium or ventricle of a heart of an object to be examined.

FIG. 5 is a flow chart which shows an operation of the system according to the embodiment.

### DETAILED DESCRIPTION

According to one embodiment, an X-ray diagnostic system is provided. The system is provided with an X-ray irradiation section, an X-ray detection section, an image data generation unit, a position detecting unit and a control section. The X-ray irradiation section radiates an X-ray to an object to be diagnosed. The X-ray detection section detects the X-ray radiated from the X-ray irradiation section and transmitted through the object so as to generate X-ray transmission data. The image data generation unit generates image data based on the X-ray transmission data. The position detecting unit detects position of a catheter inserted into the object. The control section switches X-ray irradiation conditions to be irradiated from the X-ray irradiation section, based on positional data detected by the position detecting unit and electrocardiogram data of the object.

According to another embodiment, an X-ray diagnostic system is provided. The system is provided with an X-ray irradiation section, an X-ray detection section, an image data generation unit, a position detecting unit, a data storage unit and a control section. The X-ray irradiation section radiates an X-ray to an object to be diagnosed. The X-ray detection section detects the X-ray radiated from the X-ray irradiation section and transmitted through the object so as to generate X-ray transmission data. The image data generation unit generates image data based on the X-ray transmission data. The position detecting unit detects position of a catheter inserted into the object. The data storage unit stores electrocardiogram data of the object and positional data of a past position detected by the position detecting unit at each phase of the electrocardiogram data.

The control section switches X-ray irradiation conditions to be irradiated from the X-ray irradiation section, based on positional data of a current position detected by the position detecting unit and positional data of a past position stored in the data storage unit. The positional data of the past position represents the same phase as that of the electrocardiogram data outputted when the current position detected.

According to further another embodiment, an X-ray diagnostic system is provided. The system is provided with an X-ray irradiation section, a voltage supply section, an X-ray detection section, an image data generation unit, a data storage unit, a determination unit and a system control unit.

The X-ray irradiation section radiates an X-ray to an object to be diagnosed. The voltage supply section supplies a controlled voltage to the X-ray irradiation section. The X-ray detection section detects the X-ray radiated from the X-ray irradiation section and transmitted through the object so as to generate X-ray transmission data. The image data generation unit generates image data based on the X-ray transmission data generated by the X-ray detection section. The data storage unit stores electrocardiogram data of the object and positional data of a position of a tip portion of a catheter inserted into the object. The positional data is detected by the position detecting unit at each phase of the electrocardiogram data. The determination unit determines whether a current position newly detected by the position detecting unit and a past position indicated by positional data stored in the data storage unit. The positional data of the past position represents the same phase as that of the electrocardiogram data outputted when the current position detected. The determination unit gives an instruction to control X-ray irradiation to the voltage supply section based on the determination result. The system control unit controls operations of the data storage unit and the determination unit.

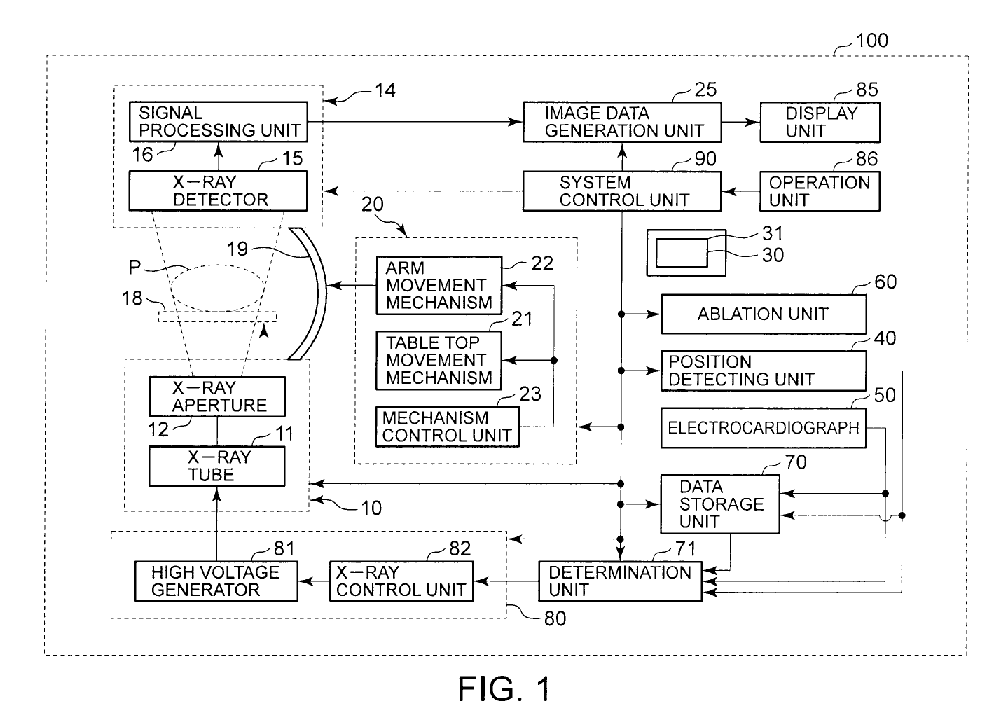

Hereinafter, a further embodiment will be described with reference to FIGS. 1 to 5. The same numerals indicate the same or similar portions respectively in FIGS. 1 to 5.

FIG. 1 is a block diagram showing a system to diagnose using an X-ray according to a first embodiment of the invention. As shown in FIG. 1, the system 100 is provided with an X-ray irradiation section 10, an X-ray detection section 14, a C-shaped arm 19 and a mechanism section 20.

The X-ray irradiation section 10 radiates an X-ray to an object P to be diagnosed which is laid on a table top 18. The X-ray irradiation section 10 detects the X-ray radiated from the X-ray irradiation section 10 and transmitted through the object, and generates an electric signal. The C-shaped arm 19 is generally called as “C-arm”. The C-shaped arm 19 holds the X-ray irradiation section 10 and the X-ray detection section 14. The mechanism section 20 performs movement of the table top 18 and the C-shaped arm 19.

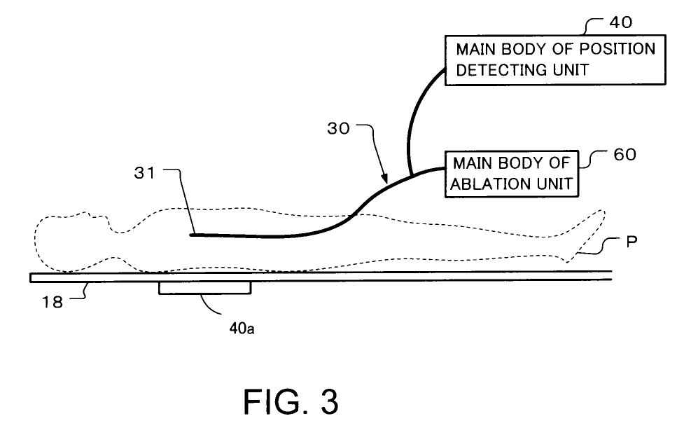

Further, the system 100 is provided with an image data generation unit 25, a position detecting unit 40, an electrocardiograph 50, and an ablation unit 60. The image data generation unit 25 produces image data based on the electric signal generated by the X-ray detection section 14. As shown in FIG. 3, the position detecting unit 40 detects the position of a catheter 30 inserted into the object P. The ablation unit 60 performs medical treatment for arrhythmia of a heart of the object P. The electrocardiograph 50 measures an electrocardiogram of the object P.

Each of detection ends of the position detecting unit 40 and the ablation unit 60 is arranged in a tip portion 31 of the catheter 30. The other end of the catheter 30 is connected to main bodies of the position detecting unit 40 and the ablation unit 60.

As shown in FIG. 1, the system 100 is further provided with the data storage unit 70, the determination unit 71, a voltage supply section 80, a display unit 85, an operation unit 86 and a system control unit 90. The data storage unit 70 stores electrocardiogram data of the object P measured with the electrocardiograph 50 and stores positional data of the catheter 30 detected by the position detecting unit 40 when the electrocardiogram is measured.

The determination unit 71 determines whether the position of the catheter 30 detected by the position detecting unit 40 corresponds to the positions indicated by the positional data stored in the data storage unit 70. The voltage supply section 80 drives the X-ray irradiation section 10. The display unit 85 displays image data generated by the image data generation unit 25. The operation unit 86 is used to input various commands, etc. The system control unit 90 performs integrated control of the above-mentioned units.

The X-ray irradiation section 10 is provided with an X-ray tube 11 and an X-ray aperture 12. The X-ray tube 11 is driven by the voltage supply section 80 and generates an X-ray. The X-ray aperture 12 is arranged between the X-ray tube 11 and the object P. The X-ray aperture 12 limits the irradiation range of the X-ray emitted from the X-ray tube 11 to irradiate the object P.

The X-ray detection section 14 is provided with an X-ray detector 15 and a signal processing unit 16. The X-ray detection section 14 is arranged opposite to the X-ray irradiation section 10. The X-ray detector 15 detects the X-ray transmitted through the object P and converts the X-ray to an electric signal. The signal processing unit 16 generates X-ray projection data base on the electric signal outputted from the X-ray detector 15.

X-ray detector 15 is a direct conversion type which converts an X-ray into an electric charge directly, for example. The X-ray detector 15 is provided with plural detection elements and a gate driver. The detection elements convert X-rays into electric charges and accumulate the converted charges respectively. The detection elements are arranged in two dimensions of a column direction and a line direction. The gate driver provides drive pulses to the detection elements to read the accumulated electric charges and outputs the read electric charges to the signal processing unit 16. The X-ray detector 15 may be an indirect conversion type which converts X-rays into electric charges after converting X-rays into light.

The signal processing unit 16 has an amplifier which converts the electric charges read from the detection elements of the X-ray detector 15 to voltage. A voltage obtained by the conversion is amplified with another amplifier. An output of the amplifier obtained by the amplification is converted into a digital signal by an A/D converter. An output signal from the A/D converter is further converted into a time series signal by a parallel-serial converter so that X-ray projection data is generated. The generated X-ray projection data is outputted to the image data generation unit 25.

The mechanism section 20 is provided with a table top movement mechanism 21, an arm movement mechanism 22 and a mechanism control unit 23. The table top movement mechanism 21 moves a table top 18 on which the object P is sustained. The table top movement mechanism 21 allows the table top 18 to move in a longitudinal direction, in a width direction or in an up and down direction so as to perform X-ray fluoroscopy and photography of the object P. The arm movement mechanism 22 moves the C-shaped arm 19 in order to set up the angle and position of the X-ray irradiation section 10 and the X-ray detection section 14 to the object P. The mechanism control unit 23 controls movement of the table top movement mechanism 21 and the arm movement mechanism 22.

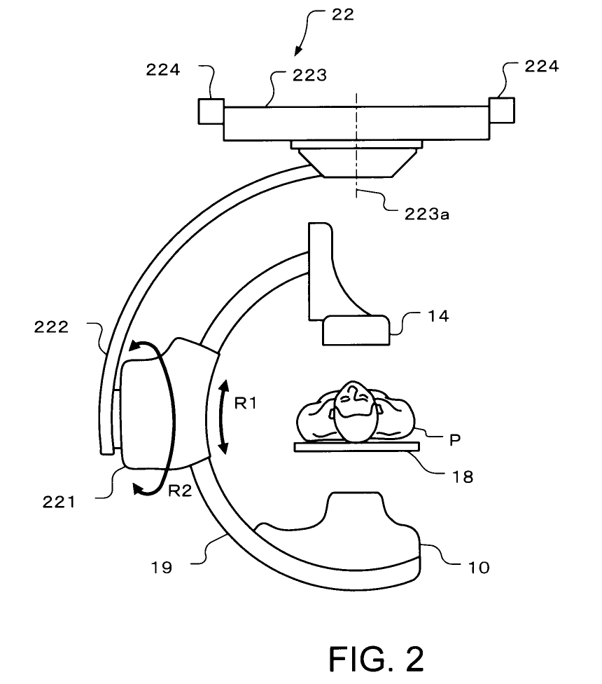

FIG. 2 shows an example of the arm movement mechanism 22. One end of the C-shaped arm 19 holds the X-ray irradiation section 10, and the other end of the C-shaped arm 19 holds the X-ray detection section 14. The Arm movement mechanism 22 is provided with first to third sustainers 221-223 and two guide rails 224, 224 arranged in an upper position. The guide rails 224, 224 extend in parallel with each other in a direction perpendicular to the sheet of FIG. 2. The first sustainer 221 supports the C-shaped arm 19 rotatably in a direction of an arrow R1. The second sustainer 222 supports the first sustainer 221 rotatably in a direction of an arrow R2. The third sustainer 223 supports the second sustainer 222 rotatably around a vertical line 223a. The third sustainer 223 is capable of moving along the guide rails 224, 224 and in a direction perpendicular to the extended direction of the guide rails 224, 224.

The image data generation unit 25 shown in FIG. 1 generates image data based on X-ray projection data outputted from signal processing unit 16 of the X-ray detection section 14, and outputs the generated image data to the display unit 85.

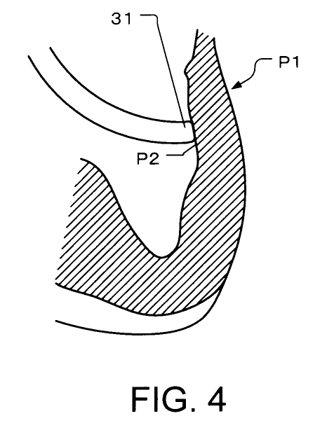

As shown in FIG. 3, in the case of performing medical treatment of arrhythmia of the object P, the tip portion 31 of the catheter 30 is inserted from a thick blood vessel, such as a femoral artery or femoral vein positioned at a leg base of the object P. The tip portion 31 of the inserted catheter 30 is sent into a heart P1 of the object P through the inside of the blood vessel. As shown in FIG. 4, the tip portion 31 of the catheter 30 is inserted so that the tip portion 31 may contact various positions of an inner wall P2 of an atrium or ventricle of the heart P1.

In medical treatment, the position detecting unit 40 detects the position of the contacted tip portion 31 of the catheter 31. An operator can find an abnormal conduction path of the heart P1 in the light of the electrical potential change indicating the position and the electrocardiogram data measured by the electrocardiograph 50. The operator burns off the abnormal conduction path by providing a high frequency current from the ablation unit 60.

The position detector 40 shown in FIGS. 1, 3 is provided with a magnetic field generator 40a, a magnetic field sensor and a processing portion to perform processing of output signal from the magnetic field sensor. The magnetic field generator 40a is placed on the back side of the table top 21 and forms a magnetic field in the object P laid on the table top 18. The magnetic field generator 40a is provided with three coils, for example. The coils are disposed apart from one another. Alternate currents are flowed in the coils.

The magnetic field sensor detects the magnetic field formed by the magnetic field generator. The magnetic field sensor may be a coil, a Hall Effect sensor, or a magnetic resistance sensor, which is arranged in the tip portion 31 of the catheter 30. The magnetic field sensor generates three magnetic fields having different frequencies with the three coils and can detect a three dimensional position of the tip portion 31 of the catheter 30. The processing portion performs processing of the output signal, which is based on the magnetic field detected by the magnetic field sensor, and draws out a coordinate system of the magnetic field sensor with respect to a fixed reference coordinate system of the magnetic field generator. Then, the processing portion generates positional data corresponding to the position of the tip portion 31 of the catheter 30. The generated positional data is outputted to the data storage unit 70 and the determination unit 71. The position detector 40 may be capable of disconnecting with the data storage unit 70, the determination unit 71 and the system control unit 90.

The electrocardiograph 50 shown in FIG. 1 is one being widely used and has a main body provided with electrodes, amplifiers connected with the electrodes and A/D converters connected with the amplifiers. The electrodes are attached onto a body surface of the object P from outside for measurement of the heart P1. The electrodes take out signals to obtain an electrocardiogram of the object P. The amplifiers amplify the signals acquired from the electrodes. The A/D converters convert the amplified signals to digital signals, and generate electrocardiogram data. The generated electrocardiogram data are outputted to the data storage unit 70 and the determination unit 71. The electrocardiograph 50 may be capable of disconnecting with the data storage unit 70 and the determination unit 71.

The ablation unit 60 has an ablation electrode and a main body having a high-frequency-current generator. The ablation electrode is arranged in the tip portion 31 of the catheter 30 so that at least a portion of itself can be exposed to the outside of the tip portion. The main body of the ablation unit 60 is placed out of the object P. The high-frequency-current generator provides a high frequency current to the ablation electrode. The high frequency current coagulates and necrotizes cells of an abnormal conduction path formed in the heart P1 of the object P. The ablation unit 60 may be capable of disconnecting with the system control unit 90.

The data storage unit 70 shown in FIG. 1 stores the electrocardiogram data measured with the electrocardiograph 50 and the positional data of the tip portion 31 of the catheter 30 detected by the position detecting unit 40, in response to a storing operation of the operation unit 86. The storing operation is performed when the tip portion 31 of the catheter 30 contacts various positions of the inner wall P2 of the atrium or ventricle of the heart P1, in order to find the abnormal conduction path of the heart P1 in medical treatment of arrhythmia.

Specifically, the electrocardiogram data is data of one or more cycles measured with the electrocardiograph 50. The positional data indicates positions of the tip portion 31 of the catheter which moves in synchronization with the heart P1 in the state where the tip portion 31 contacts various positions of the inner wall P2 of the atrium or ventricle of the heart P1. The positional data corresponds to each phase of each cycle of the electrocardiogram.

When the heart is contracted and extended, an electric signal generated from a sinus node is transmitted to the atrium and the ventricle in this order. The muscles of the atrium and the ventricle contract in the transmission order. In medical treatment of arrhythmia, the electrical potential changes of transmission courses of an electric signal are diagnosed in order. The positional data of each of the transmission courses and electrocardiogram wave data are stored in the data storage unit 70.

The determination unit 71 determines whether a position detected newly by the position detecting unit 40, i.e. a current position, corresponds to one of the positions indicated by the positional data stored in the data storage unit 70, i.e., past positions. The positional data of the past positions is positional data having the same phase as that in each cycle of the electrocardiogram which is measured by the electrocardiograph 50 when the current position is detected. Specifically, the positional data may be positional data of the tip portion 31 of the catheter 30 obtained when the operator operates starting ablation or obtained immediately before detection of the current position.

The determination unit 71 determines the current position of the tip portion 31 of the catheter 30 is same as one of the past positions, if the difference between the current position detected by the position detecting unit 40 and each of the past positions indicated by the positional data stored in the data storage unit 70 is within a predetermined acceptable range. Based on the determination result, the determination unit 71 instructs reduction of an X-ray dose to the voltage supply section 80.

The determination unit 71 determines the tip portion 31 of the catheter 30 is positioned apart from the past positions, i.e., at a new position, if the difference between the current position detected by the position detecting unit 40 and each of the past positions indicated by the positional data stored in the data storage unit 70 is out of the acceptable range. Based on the determination result, the determination unit 71 instructs generating a predetermined X-ray dose to the voltage supply section 80.

The determination unit 71 determines the tip portion 31 of the catheter 30 is at a new position, if the data storage unit 70 does not store any positional data indicating a past position which represents the same phase as that of the electrocardiogram data outputted from the electrocardiograph 50 when the current position detected. Based on the determination result, the determination unit 71 instructs generating the predetermined X-ray dose to the voltage supply section 80.

The position detecting unit 40 may detect angle of the tip portion 31 in addition to position of the tip portion 31. Further, the determination unit 71 may determine whether a change of position of the tip portion 31 exists based on the detected position and angle of the tip portion 31.

In this way, the determination unit 71 can compare a current position with each of past positions of the tip portion 31 indicated by stored positional data of the same phase as that of each cycle of electrocardiogram data, and can determine whether the current position corresponds to a position contacted with inner wall P2 of the atrium or ventricle of the heart P1 in the past, in a case where the contacted position moves periodically.

The voltage supply section 80 is provided with a high voltage generator 81 and an X-ray control unit 82 to control the high voltage generator 8. The high voltage generator 81 can drive the X-ray tube 11 of the X-ray irradiation section 10, and can generate an X-ray for fluoroscopy and an X-rays for photography having intensity larger than that of the X-ray for fluoroscopy. The X-ray control unit 82 and the determination unit 71 constitute a control section.

X-ray control unit 82 controls the high voltage generator 81 based on irradiation conditions such as tube voltage, tube current, pulse width, and pulse rate, i.e. number of times of X-ray irradiation per unit time, which are provided from the system control unit 90. Under the control, the X-rays for fluoroscopy and for photography are irradiated from X-ray tube 11 to the object P, continuously or intermittently in accordance with a predetermined pulse rate.

In medical treatment of arrhythmia, the irradiation conditions of the X-rays radiated from the X-ray irradiation section 10 are changed based on a determination result obtained from the determination unit 71. Specifically, the X-ray for fluoroscopy may be irradiated intermittently at a predetermined pulse rate by an instruction of generating the predetermined X-ray dose from the determination unit 71, when change of position of the tip portion 31 is determined based on the determination result.

Further, the X-rays for fluoroscopy are intermittently irradiated at a pulse rate lower than the predetermined pulse rate, or the irradiation of X-rays is stopped, by an instruction of reducing the X-ray dose from the determination unit 71, when any change of position of the tip portion 31 is not determined based on the determination result.

An X-ray of a predetermined dose may be radiated for fluoroscopy continuously according to an instruction of generating the predetermined X-ray dose from the determination unit 71.

Further, an X-ray may be radiated for fluoroscopy under irradiation conditions indicating an X-ray dose lower than the predetermined dose, or irradiation of the X-ray may be stopped according to an instruction of reducing the X-ray dose from the determination unit 71.

The display unit 85 is provided with a liquid crystal panel or a CRT monitor, and displays image data outputted from the image data generation unit 25.

The operation unit 86 is provided with an input device such as a key board, a trackball, a joystick or a mouse. The operation unit 86 is used for input operation to set up examination information, such as information relating to the object P to be examined including a full name and an ID number of the object, a position and an angle of the X-ray irradiation section 10 and the X-ray detection section 14, and a position of the table top 18. Further, the operation unit 86 is used for input operation to set up X-ray irradiation conditions and to set up and select various conditions relating to display.

The system control unit 90 is provided with a CPU and a memory circuit. The system control unit 90 stores inputted information temporarily. Based on the stored inputted information, the system control unit 90 controls the X-ray irradiation section 10, the X-ray detection section 14, the mechanism section 20, the image data generation unit 25, the position detecting unit 40, the ablation unit 60, the data storage unit 70, the determination unit 71, and the voltage supply section 80, and controls the system 100 as a whole.

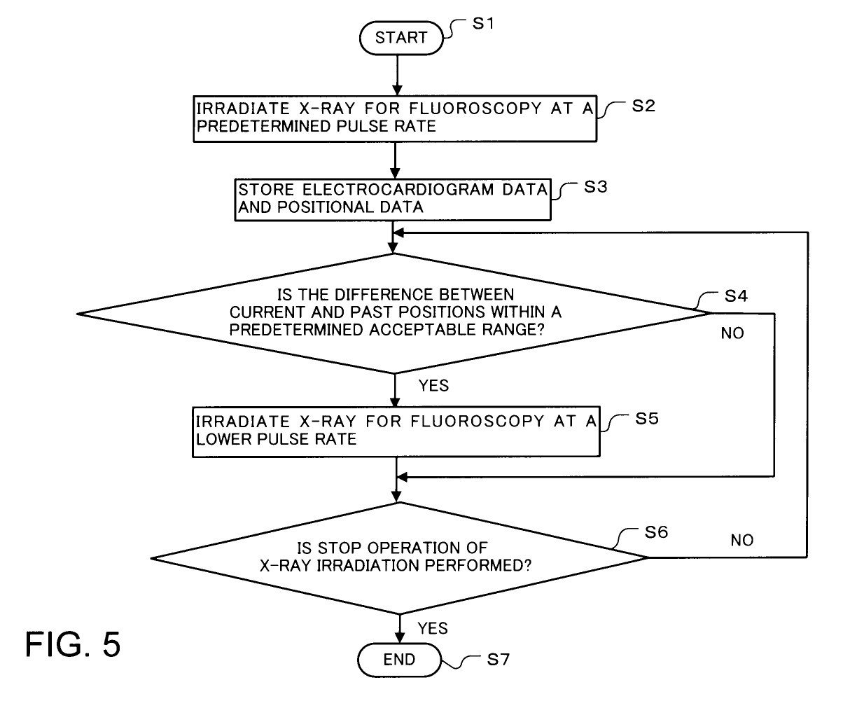

Hereinafter, an operation example of the system 100 will be explained. FIG. 5 is a flow chart showing the operation example. The operation example is executed when a medical treatment of arrhythmia is performed for the object P laid on the table top 18 shown in FIG. 1.

An operator performs an input operation to set up irradiation conditions using the operation unit 86. Then, the system 100 starts to operate when the operator performs an input operation to start an X-ray fluoroscopy by using the operation unit 86 (Step S1).

The system control unit 90 instructs start of operation of the X-ray irradiation section 10, the X-ray detection section 14, the mechanism section 20, the image data generation unit 25, the position detecting unit 40, the ablation unit 60, the data storage unit 70, the determination unit 71 and the voltage supply section 80. The magnetic field generator of the position detecting unit 40 is fixed to the table top 18, for example. The magnetic field generator forms a magnetic field in the object P laid on table top 18.

The X-ray control unit 82 of the voltage supply section 80 controls the high voltage generator 81, so as to radiate an X-ray for fluoroscopy at a predetermined pulse rate from the X-ray irradiation section 10 based on the irradiation conditions provided from the system control unit 90. The high voltage generator 81 drives X-ray irradiation section 10. The X-ray irradiation section 10 radiates an X-ray for fluoroscopy to the object P laid on table top 18 at the predetermined pulse rate (Step S2).

The X-ray detection section 14 detects the X-ray penetrated the object P, and produces X-ray projection data. Further, the X-ray detection section 14 outputs the generated X-ray projection data to the image data generation unit 25. The image data generation unit 25 generates image data based on the X-ray projection data outputted from the X-ray detection section 14, and outputs the generated image data to the display unit 85.

Thus, the X-ray is irradiated to the object P at the above-mentioned predetermined pulse rate, before an abnormal conduction path of the heart is found. By the irradiation, a clear image can be displayed on the display unit 85. A current position of the tip portion 31 of the catheter 30 can be checked easily referring to the clear image.

An operator such as a medical specialist of the heart inserts the catheter 30 into a thick blood vessel at a leg base of the object P, for example. Then, the operator operates the catheter 30 and sends the tip portion 31 into the heart, referring to an image of the tip portion 31 of the catheter 30 contained in the image displayed on the display unit 85.

The operator causes the tip portion 31 of the catheter 30 to contact with various portions of the inner wall P2 of the atrium or ventricle of the heart P1, in order to find an abnormal conduction path. The position detecting unit 40 detects a position of the tip portion 31 contacted with the inner wall P2 of the atrium or ventricle of the heart P1, and outputs positional data to the determination unit 71. The electrocardiograph 50 measures electrocardiogram data of the object P when the position of the tip portion 31 is detected, and outputs the electrocardiogram data to the determination unit 71.

The operator performs operation to renew the position of the tip portion 31 and the electrocardiogram data through the operation unit 86, each time the tip portion 31 contacts the inner wall P2. By the renewal operation, the data storage unit 70 stores electrocardiogram data of one or more cycles outputted from the electrocardiograph 50 and positional data of the tip portion 31 which is detected from the position detecting unit 40 and which corresponds to each phase of the electrocardiogram data (Step S3).

Then, the operator performs operation causing the tip portion 31 to contact the inner wall P2 of the atrium or ventricle in order to burn off the abnormal conduction path of the heart. By the operation, the determination unit 71 determines whether a current position detected by the position detecting unit 40 corresponds to one of the past positions indicated by the positional data stored in the data storage unit 70. The data of the past positions are positional data which is detected from the position detecting unit 40 and which corresponds to each phase of the electrocardiogram data.

If the difference between the current position detected by the position detecting unit 40 and each of the past positions indicated by the positional data stored in the data storage unit 70 is within a predetermined acceptable range (“YES” of Step S4), the determination unit 71 determines the current position of the tip portion 31 of the catheter 30 corresponds to one of the past positions. Based on the determination result, the determination unit 71 instructs reduction of an X-ray dose to the voltage supply section 80.

If the difference between the current position detected by the position detecting unit 40 and each of the past positions indicated by the positional data stored in the data storage unit 70 is out of the predetermined acceptable range (“NO” of Step S4), the determination unit 71 determines the current position of the tip portion 31 of the catheter 30 does not correspond to any of the past positions. Thus, the determination unit 71 determines the current position as a new position apart from any of the past positions. Based on the determination result, the determination unit 71 instructs generating the predetermined X-ray dose to the voltage supply section 80. The X-ray is irradiated from the X-ray irradiation section 10 at the above-mentioned pulse rate, also in the case where the data storage unit 70 does not store any positional data indicating a past position which represents the same phase as that of the electrocardiogram data outputted when the current position detected.

When the current position of the tip portion 31 is a new position apart from any of the past positions contacted in the past, it is necessary to display a clear image on the display unit 85 and to check the current position of the tip portion 31, referring to the displayed clear image. Therefore, the predetermined pulse rate described above is maintained. The operator can check the position of the tip portion 31 of the catheter through the clear image easily.

After the difference between the current position and one of the past positions is determined to be within the predetermined acceptable range (“YES” of Step S4), the X-ray control unit 82 controls the high voltage generator 81 so as to cause an X-ray for fluoroscopy to irradiate from the X-ray tube 11 to the object P laid on table top 18 at a low pulse rate (Step S5).

When the current position of the tip portion 31 corresponds to one of the past positions, small necessity is represented to check the current position referring to the image displayed on the display unit 85. Therefore, the pulse rate can be lowered, and the X-ray dose can be reduced to irradiate the object P.

The operator operates the ablation unit 60 through the operation unit 86 to perform an ablation operation. The ablation unit 60 provides a high frequency current from the high-frequency-current generator to the ablation electrode arranged in the tip portion 31 of the catheter 30. The high frequency current coagulates and necrotizes the abnormal conduction path formed in the inner wall P2 of the atrium or ventricle of the heart P1.

After the difference between the current position and one of the past positions is determined to be within the predetermined acceptable range (“YES” of Step S4), or after Step S5, it is determined whether the operator commands to end X-ray fluoroscopy through the operation unit 86. If the command is performed (“Yes” of Step S6), X-ray irradiation is stopped (Step S7). If the command is not performed (No of Step S6), the flow returns to Step S4.

When the operator performs the instruction to end the X-ray fluoroscopy through the operation unit 86, the system control unit 90 instructs stop of operation to the X-ray irradiation section 10, the X-ray detection section 14, the mechanism section 20, the image data generation unit 25, the position detecting unit 40, the ablation unit 60, the data storage unit 70, the determination unit 71 and the voltage supply section 80. As a result, the system 100 ends to operate.

According to the embodiment described above, the data storage unit 70 stores electrocardiogram data of the object P, and positional data indicating the position of the tip portion 31 of the catheter and corresponding to each phase of the electrocardiogram data. The determination unit 71 determines the difference between a current position of the tip portion 31 detected by the position detecting unit 40 and past positions. The past positions are shown by the positional data stored in the data storage unit 70 and correspond to each phase of the electrocardiogram data. Based on the determination, X-ray irradiation conditions can be switched to radiate X-ray from the X-ray irradiation section 10.

When the difference between the current position and any of the past positions is determined to be out of a predetermined acceptable range, an X-ray can be irradiated from the X-ray irradiation section 10 at the above-mentioned pulse rate in order to make a displayed image clear. By such an X-ray irradiation, the position of the tip portion 31 can be checked easily.

On the other hand, when the difference between the current position and one of the past positions is within a predetermined acceptable range, an X-ray can be irradiated from X-ray irradiation section 10 at a pulse rate lower than the predetermined pulse rate mentioned above. As a result, X-ray dose can be reduced to irradiate the object P so that radioactive contamination of the object P can be decreased.

While-certain-embodiments have been described, these embodiments have been presented by way of example only and are not intended to limit the scope of the inventions. Indeed, the novel systems described herein may be embodied in a variety of other forms; furthermore, various omissions and substitutions and changes in the form of the systems described herein may be made without departing from the spirit of the inventions. The accompanying claims and their equivalents are intended to cover such for ms or modifications as would fall within the scope and spirit of the inventions.

What is claimed is:

1. An X-ray diagnostic system, comprising:
an X-ray irradiation section configured to radiate an X-ray to an object to be diagnosed;
an X-ray detection section configured to detect the X-ray radiated from the X-ray irradiation section and transmitted through the object so as to generate X-ray transmission data;
an image data generation unit configured to generate image data based on the X-ray transmission data;
a position detecting unit configured to detect position of a tip portion of a catheter inserted into the object; and
a control section configured to switch X-ray irradiation conditions to be irradiated from the X-ray irradiation section, based on positional data detected by the position detecting unit and electrocardiogram data of the object.

2. An X-ray diagnostic system according to claim 1, wherein the control section causes X-ray to irradiate from the X-ray irradiation section based on a predetermined irradiation condition, when the difference between a current position and a past position respectively detected by the position detecting unit is out of a predetermined acceptable range.

3. An X-ray diagnostic system according to claim 2, wherein the control section causes X-ray to radiate from the X-ray irradiation section based on an irradiation condition with an X-ray dose smaller than that of the predetermined irradiation condition, or to stop radiating from the X-ray irradiation section, when the difference between a current position and a past position respectively detected by the position detecting unit is within the predetermined acceptable range.

4. An X-ray diagnostic system according to claim 2, wherein the control section causes X-ray to radiate from the X-ray irradiation section based on the predetermined irradiation condition when any positional data indicating a past position is not stored, the positional data indicating the past position representing the same phase as that of the electrocardiogram data outputted when the current position is detected.

5. An X-ray diagnostic system according to claim 1 wherein the position of the tip portion of the catheter inserted into the object is a position to contact an inner wall of an atrium or a ventricle of a heart of the object.

6. An X-ray diagnostic system, comprising:
an X-ray irradiation section configured to radiate an X-ray to an object to be diagnosed;
an X-ray detection section configured to detect the X-ray radiated from the X-ray irradiation section and transmitted through the object so as to generate X-ray transmission data;
an image data generation unit configured to generate image data based on the X-ray transmission data;
a position detecting unit configured to detect position of a tip portion of a catheter inserted into the object;
a data storage unit configured to store electrocardiogram data of the object and positional data of a past position detected by the position detecting unit at each phase of the electrocardiogram data; and
a control section configured to switch X-ray irradiation conditions to be irradiated from the X-ray irradiation section, based on positional data of a current position detected by the position detecting unit and positional data of a past position stored in the data storage unit, the positional data of the past position representing the same phase as that of the electrocardiogram data outputted when the current position detected.

7. An X-ray diagnostic system according to claim 6, wherein the control section causes X-ray to radiate from the X-ray irradiation section based on a predetermined irradiation condition, when the difference between the current position and the past position respectively detected by the position detecting unit is out of a predetermined acceptable range.

8. An X-ray diagnostic system according to claim 7, wherein the control section causes X-ray to radiate from the X-ray irradiation section based on an irradiation condition with an X-ray dose smaller than that of the predetermined irradiation condition, or to stop radiating from the X-ray irradiation section, when the difference between the current position and the past position respectively detected by the position detecting unit is within the predetermined acceptable range.

9. An X-ray diagnostic system according to claim 7, wherein the control section causes X-ray to radiate from the X-ray irradiation section based on the predetermined irradiation condition when any positional data indicating a past position is not stored, the positional data indicating the past position representing the same phase as that of the electrocardiogram data outputted when the current position is detected.

10. An X-ray diagnostic system according to claim 6 wherein the position of the tip portion of the catheter inserted into the object is a position to contact an inner wall of an atrium or a ventricle of a heart of the object.

11. An X-ray diagnostic system, comprising:
an X-ray irradiation section configured to radiate an X-ray to an object to be diagnosed;
a voltage supply section configured to supply a controlled voltage to the X-ray irradiation section;
an X-ray detection section configured to detect the X-ray radiated from the X-ray irradiation section and transmitted through the object so as to generate X-ray transmission data;
an image data generation unit configured to generate image data based on the X-ray transmission data generated by the X-ray detection section;
a data storage unit configured to store electrocardiogram data of the object and positional data of a position of a tip portion of a catheter inserted into the object, the positional data being detected by the position detecting unit at each phase of the electrocardiogram data;
a determination unit configured to determine whether a current position newly detected by the position detecting unit and a past position indicated by positional data stored in the data storage unit, the positional data of the past position representing the same phase as that of the electrocardiogram data outputted when the current position detected, and the determination unit being configured to give an instruction to control X-ray irradiation to the voltage supply section based on the determination result; and
a system control unit configured to control operations of the data storage unit and the determination unit.

12. An X-ray diagnostic system according to claim 11, wherein the determination unit controls the X-ray irradiation so that the X-ray dose to be irradiated from the X-ray irradiation section can be reduced.

13. An X-ray diagnostic system according to claim 12, wherein the reduction of the X-ray dose is performed by irradiating the X-ray irradiation at a smaller pulse rate.

14. An X-ray diagnostic system according to claim 11, wherein the determination unit controls the X-ray irradiation so that the X-ray irradiation from the X-ray irradiation section can be stopped.

15. An X-ray diagnostic system according to claim 11,
wherein the voltage supply section causes X-ray to radiate from the X-ray irradiation section based on a predetermined X-ray irradiation condition when the difference between the current position and the past position respectively detected by the position detecting unit is out of a predetermined acceptable range, the past position being positional data stored in the data storage unit and representing the same phase as that of the electrocardiogram data outputted when the current position detected, and
wherein the voltage supply section causes X-ray to radiate from the X-ray irradiation section based on an irradiation condition with a dose smaller than that of the predetermined irradiation condition, or to stop radiating from the X-ray irradiation section, when the difference between the current position and the past position respectively detected by the position detecting unit is within the predetermined acceptable range.

16. An X-ray diagnostic system according to claim 15, wherein the X-ray is irradiated from the X-ray irradiation section based on the predetermined irradiation condition when any positional data indicating a past position is not stored, the positional data indicating a position representing the same phase as that of the electrocardiogram data outputted when the current position is newly detected.

17. An X-ray diagnostic system according to claim 11 wherein the position of the tip portion of the catheter inserted into the object is a position to contact an inner wall of an atrium or a ventricle of a heart of the object.

18. An X-ray diagnostic system according to claim 11, further comprising a position detecting unit configured to detect position of a tip portion of a catheter, wherein the position detecting unit is detachably connected with the data storage unit and the determination unit, and provides a detected positional data to the data storage unit and the determination unit.
